(19) United States
(12) Patent Application Publication
(10) Pub. No.: US 2022/0411825 A1
(43) Pub. Date: Dec. 29, 2022

(54) METHOD OF ENGINEERING HYPOIMMUNOGENIC MUSCLE PRECURSOR CELLS

(71) Applicant: VITA THERAPEUTICS, INC., Baltimore, MD (US)

(72) Inventors: Douglas Falk, Baltimore, MD (US); Peter Andersen, Baltimore, MD (US)

(73) Assignee: VITA THERAPEUTICS, INC., Baltimore, MD (US)

(21) Appl. No.: 17/778,728

(22) PCT Filed: Nov. 20, 2020
(86) PCT No.: PCT/US2020/061602

§ 371 Date: May 20, 2022

Related U.S. Application Data
(60) Provisional application No. 62/938,053, filed on Nov. 20, 2019.

(51) Int. Cl. C12N 15/90; C12N 5/077; C12N 15/11; C12N 9/22; A61K 35/34

(52) U.S. Cl. CPC C12N 15/907; C12N 5/0659; C12N 15/11; C12N 9/22; A61K 35/34; C12N 2310/20; C12N 2800/80

(57) ABSTRACT

Embodiments of the invention relate to stem cells having conditional immune-evasion capabilities, methods of preparing cells and methods for using these stem cells to repair muscle tissue.

### CROSS-REFERENCE OF RELATED APPLICATION

This application claims priority to U.S. Provisional Application No. 62/938,053 filed Nov. 20, 2019; the entire contents of all of which are hereby incorporated by reference.

### BACKGROUND

### 1. Field of the Invention

The field of the currently claimed embodiments of this invention relate to stem cells having conditional immune-evasion capabilities, methods of preparing cells and methods for using these stem cells to repair muscle tissue.

### 2. Discussion of Related Art

One major roadblock in skeletal muscle cell replacement therapy based on stem cells, such as pluripotent stem cells (embryonic stem cells and induced pluripotent stem cells), is limited integration of transplanted skeletal muscle cells at the injury site. Most cell transplantations require multiple cell injections because patients need new cells continuously in their lives. A second major barrier is the risk of rejection by the recipient's immune system and/or the risk of tumor formation. Thus, new methods for producing therapies that target muscle diseases and syndromes are needed.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a myogenic stem cell having a modulated expression of a HLA-A gene, a HLA-B gene, a HLA-C gene, a β-Microglobulin (B2M) gene, a CIITA gene, a CD74 gene, a TAP1 gene, a MIC-1 gene, a MIC-2 gene, a CD24 gene, an HLA-G gene, and an HLA-E gene relative to a wildtype stem cell.

An embodiment of the invention relates to a method of preparing a hypoimmunogenic stem cell, including: reducing expression of at least one of an HLA-A gene, an HLA-B gene, an HLA-C gene, a β-Microglobulin (B2M) gene, a CIITA gene, a CD74 gene, a TAP1 gene, a MIC-1 gene, and a MIC-2 gene in a stem cell; and conditionally expressing at least one of a CD24 gene, an HLA-G gene, and an HLA-E gene in the stem cell; where the reducing the expression of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene and conditionally expressing the at least one of the CD24 gene, the HLA-G gene, and the HLA-E gene results in the hypoimmunogenic stem cell.

An embodiment of the invention relates to a method of treating a subject in need thereof including administering to the subject the myogenic stem cell described above.

An embodiment of the invention relates to a composition including the myogenic stem cell described above.

### BRIEF DESCRIPTION OF THE DRAWINGS

FIGS. 1A-1D depict a general schematic showing how immune evasion is achieved according to an embodiment of the invention.

FIG. 2 is a schematic showing a gene alteration strategy and role during immunity according to an embodiment of the invention.

FIGS. 3A-3C show the muscle-specific conditional expression of immunomodulatory factors according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Definitions are included herein for the purpose of understanding the present subject matter and the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used throughout, the terms “conditionally expressed” or “conditional expression” refer to the engineered expression of a gene in a stem cell such that the gene is expressed only during certain stages of the stem cell's differentiation and/or within certain tissues. In such cases, expression of the gene is reduced or inhibited in other stages of the stem cell's differentiation and/or in other tissues. In some embodiments, the stem cell is engineered to express the gene at a higher level, or “over-express” the gene, during certain stages of the stem cell's differentiation and/or within certain tissues as compared to the level of expression of that gene in a wild-type or non-engineered version of the stem cell.

In some embodiments, a gene is conditionally overexpressed in a stem cell relative to wild-type or native levels of expression of the gene in a wild-type of native version of the stem cell. In some embodiments, the gene to be conditionally overexpressed is regulated by a muscle precursor and/or a muscle specific promotor. In such embodiments, the promotor is active during specific differentiation stages of the stem cell and inactive during other differentiation stages of the stem cell. By way of non-limiting example, such a promotor is active in muscle cells including myogenic stem cell/satellite cells, myoblasts and muscle fibers, and is inactive in non-muscle lineages including but not limited to fibroblasts and adipocytes. Non-limiting examples of such promotors are provided below in Table 1.

TABLE 1
Example promotors for conditional expression of a gene

| | | | | Tissue | |
|---|---|---|---|---|---|
| Promoter | Name | Aliases | Type | expression | Cell Type |
| PAX7 | Paired box 7 | HUP1, | Transcription | Muscle | Satellite |
| | | PAX7B, | factor | tissue | cells, |
| | | RMS2, | | | myoblasts |
| | | MYOSCO | | | |
| MYF4 | Myogenic | MYOG | Transcription | Muscle | Myoblasts, |
| | factor 4 | | factor | tissue | Myofibers |
| ANFRD1 | Cardiac | ALRP, | Transcription | Muscle | Myofibers |
| | ankyrin | CARP | factor | tissue | |
| | repeat | | | | |
| | protein | | | | |
| MYOD1 | Myoblast | MYF3, PUM | Transcription | Muscle | Myoblasts |
| | determination | | factor | tissue | |
| | factor 1 | | | | |
| Myf5 | Myogenic | EORVA | Transcription | Muscle | Satellite |
| | factor 5 | | factor | tissue | cells |

A used throughout, the term “modulated expression” refers to a change in expression which can include increasing expression (or that the gene is over expressed) or decreasing expression (or that the gene is under expressed). Gene expression can also be decreased by deleting the gene, which is considered as a modulated expression of the gene.

As used throughout, the terms “hypoimmunogenic” or “immune-evasive” refer to a stem cell engineered to have a decreased immunogenicity or to otherwise avoid provocation of an immune response. In some embodiments, the hypoimmunogenic or immune-evasive properties of the engineered stem cell are conditional and the stem cell has a decreased immunogenicity only during certain non-muscle differentiation stages. In some embodiments, the conditional immune-evasive properties of the stem cell aids with the proper differentiation and maturation of the stem cell to a desired cell or tissue type. In such embodiments, the stem cell is further engineered to inactivate its immune-evasive properties if the cell fails to differentiate or mature into the desired cell or tissue type so that an immune response can be mounted against the stem cell, thus preventing unintended differentiation and aberrant proliferation of the stem cell.

In embodiments, a “muscular disorder” or “muscle disease” or “muscle syndrome” or “muscle condition” or “myopathy” is a disorder that results in increasing weakening and breakdown of skeletal muscles over time. For example, muscular dystrophy (MD) contains at least thirty different genetic disorders that are usually classified into nine main categories or types. MD refers to a group of hereditary, progressive, degenerative disorders characterized by progressive muscle weakness, defects in muscle proteins, and the destruction of muscle fibers and tissue over time. In many cases, the histological picture shows variation in fiber size, muscle cell necrosis and regeneration, and often proliferation of connective and adipose tissue. The diseases primarily target the skeletal or voluntary muscles. However, muscles of the heart and other involuntary muscles are also affected in certain forms of muscular dystrophy.

The present invention can be used in the treatment of degenerative muscular wasting disorders as well as volumetric muscle loss. Examples of degenerative muscular wasting disorders include Muscular Dystrophies, Myopahties, Mitochondrial Diseases, Soft Tissue Sarcomas, Ion Channel Diseases, Cachexia, and Sarcopenia. Volumetric muscle loss includes muscle loss due to traumatic injury or surgery. Surgery can be, for example any procedure in which muscle tissue is removed, for example a surgery due to cancer such as rhabdomyosarcoma.

Muscular Dystrophies are a group of inherited diseases that cause progressive weakness and loss of muscle mass. In muscular dystrophy, abnormal genes (mutations) interfere with the production of proteins needed to form healthy muscle. For the most part, the satellite cells within muscular dystrophy patients lack the proteins necessary for muscle production. Satellite cells or myogenic stem cells according to the invention are fully functional and healthy. They will undergo asymmetric cell division and give rise to myoblasts that will fuse with the hosts myoblasts to restore damaged muscle and create new muscle. The inventive cells have a curative long term effect on continuous muscle regeneration. Examples of muscular dystrophies include Muscular Dystrophies include Limb-girdle muscular dystrophies (LGMD), Becker muscular dystrophy (BMD), Congenital muscular dystrophies (CMD), (for example, Bethlem CMD, Fukuyama CMD, Muscle-eye-brain diseases (MEBs), Rigid spine syndromes, Ullrich CMD, Walker-Warburg syndromes (WWS)), Duchenne muscular dystrophy (DMD), Emery-Dreifuss muscular dystrophy (EDMD), Facioscapulohumeral muscular dystrophy (FSHD), Myotonic dystrophy (DM), and Oculopharyngeal muscular dystrophy (OPMD).

Myopathies are a group of inherited muscle diseases associated with the loss of muscle function and strength. It is unknown what causes inflammatory myopathies, however, the belief is that something goes wrong in the immune system, which leads to an attack of the muscle cells. Causes also include infection, muscle injury due to medicine, inherited diseases that affect muscle function, disorders of electrolyte levels, and thyroid disease. The satellite cells or myogenic stem cells according to the invention will produce muscle that evade the immune system. Therefore, the cells will not be targeted by the immune system and can repair muscle function and strength to patients. Myopathies treatable using the invention include Congenital myopathies (for example, Cap myopathies, Centronuclear myopathies, Congenital myopathies with fiber type disproportion, Core myopathies, Central core disease, Multiminicore myopathies, Myosin storage myopathies, Myotubular myopathy, and Nemaline myopathies), Distal myopathies (for example, GNE myopathy/Nonaka myopathy/hereditary inclusion-body myopathy (HIBM), Laing distal myopathy, Markesberg-Griggs late-onset distal myopathy, Miyoshi myopathy, Udd myopathy/tibial muscular dystrophy, VCP Myopathy/IBMPFD, Vocal cord and pharyngeal distal myopathy, and Welander distal myopathy), Endocrine myopathies (such as Hyperthyroid myopathy and Hypothyroid myopathy), Inflammatory myopathies (including Dermatomyositis, Inclusion-body myositis, and Polymyositis), Metabolic myopathies (for example, Acid maltase deficiency (AMD, Pompe disease), Carnitine deficiency, Carnitine palmityl transferase deficiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, and Phosphorylase deficiency (McArdle disease)), Myofibrillar myopathies (MFM), and Scapuloperoneal myopathy.

Mitochondrial Diseases are a group of inherited diseases where the disease occurs when mitochondria fail to produce enough energy for the body to function properly. This leads to muscle weakness, muscle pain, and low muscle tone. The inventive satellite cells or myogenic stem cells can significantly improve the quality of lives for patients suffering from mitochondrial diseases as they will help decrease muscle weakness by restoring muscle function and strength. Examples of Mitochondrial disease include Friedreich's ataxia (FA) and Mitochondrial myopathies such as Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, and Progressive external opthalmoplegia (PEO).

Soft Tissue Sarcomas are a group of localized soft tissue cancers. For example, Rhabdomyosarcoma (RMS), is an aggressive and highly malignant form of cancer that develops from skeletal (striated) muscle cells that have failed to fully differentiate. It is typically treated via surgery and chemotherapy and tends to yield a high survival rate. However, afterwards the patient is left with a very weak muscles in the affected areas because it is either surgically removed or damaged by localized radiation therapy. Injections of satellite cells or myogenic stem cells according to the invention could help restore muscle regeneration in the muscle areas of the patients who survive these diseases. Other soft tissue sarcomas that can result in muscle loss or removal include Angiosarcoma, Dermatofibrosarcoma protuberans, Epithelioid sarcoma, Gastrointestinal stromal tumor (GIST), Kaposi's sarcoma, Leiomyosarcoma, Liposarcoma, Malignant peripheral nerve sheath tumors, Myxofibrosarcoma, Pleomorphic sarcoma, Rhabdomyosarcoma, Solitary fibrous tumor, Synovial sarcoma, and Undifferentiated pleomorphic sarcoma

Ion Channel Diseases are a group of diseases associated with defects in ion channels which are typically marked by muscular weakness, absent muscle tone, or episodic muscle paralysis. Satellite cells or myogenic stem cells according to the invention can help restore functional muscle in these patients. Examples of Ion Channel Diseases include Andersen-Tawil syndrome, Hyperkalemic periodic paralysis, Hypokalemic periodic paralysis, Myotonia congenita (for example, Becker myotonia and Thomsen myotonia), Paramyotonia congenita, and Potassium-aggravated myotonia.

Cachexia is a complex and multifactorial disorder characterized by pathophysiological changes that alter body composition (through muscle loss), quality of life, performance status, morbidity, and mortality, with up to half of patients with cancer dying with cachexia and up to 20% of them having cachexia as the cause of death. In view of its increased prevalence, cachexia has been proposed to be a cancer comorbidity. Satellite cells or myogenic stem cells of the invention can significantly improve the quality of lives for patients suffering from cachexia as they will help restore muscle regeneration.

Sarcopenia is a condition characterized by loss of skeletal muscle mass and function. Although it is primarily a disease of the elderly, its development may be associated with conditions that are not exclusively seen in older persons. Sarcopenia is a syndrome characterized by progressive and generalized loss of skeletal muscle mass and strength and it is strictly correlated with physical disability, poor quality of life and death. The loss of satellite cells overtime is believed to be one of the leading causes that attributes to sarcopenia. Injections of satellite cells or myogenic stem cells according to the invention can significantly slow the progressiveness of sarcopenia.

A particularly lethal type of MD is Duchenne muscular dystrophy (DMD) which typically affects males beginning around the age of four. Other types include Becker muscular dystrophy, facioscapulohumeral muscular dystrophy, and myotonic dystrophy. They are due to mutations in genes that are involved in making muscle proteins. This can occur due to either inheriting the defect from one's parents or the mutation occurring during early development. Disorders may be X-linked recessive, autosomal recessive, or autosomal dominant. Additional examples of muscular dystrophies include Limb Girdle Muscular Dystrophy, Oculopharyngeal muscular dystrophy, Emery-Dreifuss muscular dystrophy, Fukuyama-type congenital muscular dystrophy, Miyoshi myopathy, Ullrich congenital muscular dystrophy, Steinert Muscular Dystrophy.

Duchenne Muscular Dystrophy (DMD) is the most common inherited lethal childhood muscular dystrophy, affecting about 1 in 3000 males. Children with DMD usually become wheelchair bound by the age of 11 or 12 years and affected individuals usually die in the second or third decade of life. DMD originates from mutations in the dystrophin gene located on the X chromosome (Xp21), leading to loss of dystrophin protein with attendant muscle fiber destruction. Although the role of the dystrophin protein in maintaining skeletal myofiber integrity is generally well recognized, the exact mechanism that leads to myofiber destruction and loss in dystrophic muscle is not well understood. The discovery of the dystrophin gene and the subsequent characterization of the protein product have established dystrophin as an integral sarcolemmal protein, linking the muscle sarcomere and cytoskeleton to the surrounding extracellular matrix. The localization of dystrophin is synonymous with maintaining muscle integrity and its absence (as evidenced in DMD) leads to membrane fragility, contraction induced myofiber damage, and death.

Current DMD Treatment There is no known cure for DMD, and an ongoing medical need has been recognized by regulatory authorities. Treatment is generally aimed at controlling the onset of symptoms to maximize the quality of life which can be measured using specific questionnaires, and include:

Corticosteroids such as prednisolone and deflazacort lead to short-term improvements in muscle strength and function up to 2 years. Corticosteroids have also been reported to help prolong walking.

2 agonists that increase muscle strength, but do not modify disease progression (e.g., salbutamol (e.g., albuterol) which is a β₂ agonist may be used).

Mild, nonjarring physical activity such as swimming is encouraged. Inactivity (such as bed rest) can worsen the muscle disease.

Physical therapy is helpful to maintain muscle strength, flexibility, and function.

Orthopedic appliances (such as braces and wheelchairs) may improve mobility and the ability for self-care. Form-fitting removable leg braces that hold the ankle in place during sleep can defer the onset of contractures.

Appropriate respiratory support as the disease progresses is important.

Cardiac problems may require a pacemaker.

The medication, e.g., ataluren may also be provided.

By “muscle stem cell” or “myogenic stem cell” or “myogenic progenitor cell” is meant a self-renewing mononucleate cell that produces as progeny mononucleate myoblasts, which are committed to form multinucleate myofibers via intercellular fusion. Myogenic stem cells include satellite cells, which are tissue specific myogenic stem cells that can self-renew and give rise to myoblasts, which serve the ‘building blocks’ of skeletal muscle. Encompassed herein, are muscle stem cells that produce skeletal muscle, smooth muscle, or cardiac muscle. The terms “muscle stem cell,” “myogenic stem cell,” “myogenic progenitor cell,” and “satellite cell” are used interchangeably herein.

The term “muscle cell” as used herein refers to any cell which contributes to muscle tissue. Myoblasts, satellite cells, myotubes, and myofibril tissues all contribute to muscle tissue and can be included in the term “muscle cells.” Muscle cell effects may be induced within skeletal, cardiac and smooth muscles. Muscle tissue in adult vertebrates will regenerate from satellite cells. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleated myotubes which form bundles of new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

“Muscle growth” as used herein refers to the growth of muscle which may occur by an increase in the fiber size and/or by increasing the number of fibers. The growth of muscle as used herein may be measured by A) an increase in wet weight, B) an increase in protein content, C) an increase in the number of muscle fibers, or D) an increase in muscle fiber diameter. An increase in growth of a muscle fiber can be defined as an increase in the diameter where the diameter is defined as the minor axis of ellipsis of the cross section.

“Myogenic” cells as described herein are those cells that are related to the origin of muscle cells or fibers. Various molecular markers are known to be specific for the middle and late stages of myogenic differentiation. For example, in C2C12 cells, myosin and MRF4 mark the late stages of myogenesis and are largely restricted to myotubes, whereas myogenin and nestin mark the middle stages of myogenesis and are found in all myotubes and in many committed myoblasts.

“Atrophy” or “wasting” of muscle as used herein refers to a significant loss in muscle fiber girth. By significant atrophy is meant a reduction of muscle fiber diameter in diseased, injured or unused muscle tissue of at least 10% relative to undiseased, uninjured, or normally utilized tissue.

The term “disease” refers to any deviation from the normal health of a mammal and includes a state when disease symptoms are present, as well as conditions in which a deviation (e.g., muscle dysfunction or muscle disorder) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

Embodiments of the invention relate to a stem cell having conditional immune-evasion capabilities, and to methods for using the stem cell to repair muscle tissue.

An embodiment of the invention relates to a myogenic stem cell having a modulated expression of a HLA-A gene, a HLA-B gene, a HLA-C gene, a β-Microglobulin (B2M) gene, a CIITA gene, a CD74 gene, a TAP1 gene, a MIC-1 gene, a MIC-2 gene, a CD24 gene, an HLA-G gene, and an HLA-E gene relative to a wildtype stem cell.

An embodiment of the invention relates to the myogenic stem cell above, where the modulated expression of at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene is reduced relative to a wildtype stem cell.

An embodiment of the invention relates to the myogenic stem cell above, where an expression of the HLA-A gene, the HLA-B gene, the HLA-C gene, the β-Microglobulin (B2M) gene, the CIITA gene, the CD74 gene, the TAP1 gene, and the MIC-1 gene is inhibited.

An embodiment of the invention relates to the myogenic stem cell above, where the HLA-A gene, the HLA-B gene, the HLA-C gene, the β-Microglobulin (B2M) gene, the CIITA gene, the CD74 gene, the TAP1 gene, the MIC-1 gene, and the MIC-2 gene are deleted.

An embodiment of the invention relates to the myogenic stem cell above, where at least one of the CD24 gene, the HLA-G gene, and the HLA-E gene is conditionally over-expressed relative to a wildtype stem cell.

An embodiment of the invention relates to the myogenic stem cell above, where at least one of the CD24 gene, the HLA-G gene, and the HLA-E gene is over-expressed during a first cell-differentiation stage, and where the at least one of the CD24 gene, the HLA-G gene, and the HLA-E gene has a reduced level of expression during a second cell-differentiation stage relative to the first cell-differentiation stage.

An embodiment of the invention relates to the myogenic stem cell above, where at least one of the CD24 gene, the HLA-G gene, and the HLA-E gene is regulated by a pax7 promotor, a pax3 promotor, a myh4 promotor, a Ankrd1 promotor, a myod1 promotor, and myf5 promotor, or a myog promotor.

An embodiment of the invention relates to the myogenic stem cell above, where the CD24 gene, the HLA-G gene, and the HLA-E gene are conditionally over-expressed relative to a wildtype stem cell.

An embodiment of the invention relates to the myogenic stem cell above, where the CD24 gene, the HLA-G gene, and the HLA-E gene are over-expressed during a first cell-differentiation stage, and where the CD24 gene, the HLA-G gene, and the HLA-E gene have a reduced level of expression during a second cell-differentiation stage relative to the first cell-differentiation stage.

An embodiment of the invention relates to the myogenic stem cell above, where expression of the CD24 gene, the HLA-G gene, and the HLA-E gene are regulated by a pax7 promotor, a pax3 promotor, a myh4 promotor, a Ankrd1 promotor, a myod1 promotor, and myf5 promotor, or a myog promotor.

An embodiment of the invention relates to the myogenic stem cell above, further including an increased expression of at least one of a CD46 gene, a CD59 gene, and a CD55 gene relative to a wildtype stem cell.

An embodiment of the invention relates to the myogenic stem cell above, further including an increased expression of each of the CD46 gene, the CD59 gene, and the CD55 gene relative to a wildtype stem cell.

An embodiment of the invention relates to the myogenic stem cell above, where at least one of the CD24 gene, the HLA-G gene, and the HLA-E gene is inserted into a safe harbor locus of at least one allele of the stem cell.

An embodiment of the invention relates to the myogenic stem cell above, where the safe harbor locus includes an AAVS1 locus.

An embodiment of the invention relates to the myogenic stem cell above, where the stem cell is hypoimmunogenic.

An embodiment of the invention relates to the myogenic stem cell above, further including a reduced expression of at least one of an HLA-DMA gene, an HLA-DMB gene, an HLA-DOA gene, an HLA-DOB gene, an HLA-DPA1 gene, an HLA-DPB1 gene, an HLA-DQA1 gene, an HLA-DQA2 gene, an HLA-DQB1 gene, an HLA-DQB2 gene, an HLA-DRA gene, an HLA-DRB1 gene, an HLA-DRB3 gene, an HLA-DRB4 gene, and an HLA-DRB5 gene relative to a wildtype stem cell.

An embodiment of the invention relates to the myogenic stem cell above, where at least one of the HLA-DMA gene, the HLA-DMB gene, the HLA-DOA gene, the HLA-DOB gene, the HLA-DPA1 gene, the HLA-DPB1 gene, the HLA-DQA1 gene, the HLA-DQA2 gene, the HLA-DQB1 gene, the HLA-DQB2 gene, the HLA-DRA gene, the HLA-DRB1 gene, the HLA-DRB3 gene, the HLA-DRB4 gene, and the HLA-DRB5 is deleted.

An embodiment of the invention relates to a method of preparing a hypoimmunogenic stem cell, including: reducing expression of at least one of an HLA-A gene, an HLA-B gene, an HLA-C gene, a β-Microglobulin (B2M) gene, a CIITA gene, a CD74 gene, a TAP1 gene, a MIC-1 gene, and a MIC-2 gene in a stem cell; and conditionally expressing at least one of a CD24 gene, an HLA-G gene, and an HLA-E gene in the stem cell; where the reducing the expression of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene and conditionally expressing the at least one of the CD24 gene, the HLA-G gene, and the HLA-E gene results in the hypoimmunogenic stem cell.

An embodiment of the invention relates to the method above, further including increasing expression of at least one of a CD46 gene, a CD59 gene, and a CD55 gene in the stem cell.

An embodiment of the invention relates to the method above, where reducing expression of at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene includes deletion of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene a genomic DNA of the stem cell.

An embodiment of the invention relates to the method above, where the deletion of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene from the genomic DNA of the stem cell includes use of an endonuclease.

An embodiment of the invention relates to the method above, further including reducing expression of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene in the stem cell.

An embodiment of the invention relates to the method above, where conditionally expressing at least one of the CD24 gene, the HLA-G gene, and the HLA-E gene in the stem cell includes inserting at least one of the CD24 gene, the HLA-G gene, and the HLA-E gene into a safe harbor locus of at least one allele of the stem cell.

An embodiment of the invention relates to the method above, where the safe harbor locus includes an AAVS1 locus.

An embodiment of the invention relates to the method above, where conditionally expressing at least one of the CD24 gene, the HLA-G gene, and the HLA-E gene in the stem cell includes over-expressing at least one of the CD24 gene, the HLA-G gene, and the HLA-E gene during a first cell-differentiation stage, and reducing expression of the at least one of the CD24 gene, the HLA-G gene, and the HLA-E gene during a second cell-differentiation stage relative to the first cell-differentiation stage.

An embodiment of the invention relates to the method above, where conditionally expressing at least one of the CD24 gene, the HLA-G gene, and the HLA-E gene in the stem cell includes use of a pax7 promotor, a pax3 promotor, a myh4 promotor, a Ankrd1 promotor, a myod1 promotor, and myf5 promotor, or a myog promotor.

An embodiment of the invention relates to the method above, further including conditionally expressing the CD24 gene, the HLA-G gene, and the HLA-E gene in the stem cell.

An embodiment of the invention relates to the method above, further including reducing an expression of at least one of an HLA-DMA gene, an HLA-DMB gene, an HLA-DOA gene, an HLA-DOB gene, an HLA-DPA1 gene, an HLA-DPB1 gene, an HLA-DQA1 gene, an HLA-DQA2 gene, an HLA-DQB1 gene, an HLA-DQB2 gene, an HLA-DRA gene, an HLA-DRB1 gene, an HLA-DRB3 gene, an HLA-DRB4 gene, and an HLA-DRB5 gene in the stem cell.

An embodiment of the invention relates to a method of treating a subject in need thereof including administering to the subject any of the myogenic stem cells above.

An embodiment of the invention relates to the method above, where the subject suffers from a muscular disorder.

An embodiment of the invention relates to a composition including any of the myogenic stem cells above.

In certain embodiments, the stem cell is a myogenic stem cell, in other embodiments, the stem cell is an induced Pluripotent Stem Cell (iPSC). In some embodiments, the stem cell has a) a disrupted Human leucocyte antigen-A (HLA-A), HLA-B, HLA-C, β-Microglobulin (B2M), Class II Major Histocompatibility Complex Transactivator (CIITA), Cluster of Differentiation 74 (CD74), HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, Transporter associated with Antigen Processing 1 (TAP1), Macrophage inhibitory cytokine-1 (MIC-1), and/or MIC-2; b) over-expressed CD46, CD59, CD55, and/or CD200 and c) a PD-L1, HLA-G, HLA-E and/or a CD24 gene under the control of a conditional promoter. In some such embodiments, the conditional promotor is the Pax-7 promoter, but it can be any promoter that is active only during certain stages of cell differentiation. More specifically, the promoter is active during a satellite cell stage and during a myoblast cell stage, but it turns off once fused myoblasts differentiate into muscle fiber.

An embodiment of the invention relates to a stem cell having modulated expression of the genes Human leucocyte antigen-A (HLA-A), HLA-B, HLA-C, β-Microglobulin (B2M), Class II Major Histocompatibility Complex Transactivator (CIITA), Cluster of Differentiation 74 (CD74), HLA-DMA, HLA-DMB, HLA-DOA, HLA-DOB, HLA-DPA1, HLA-DPB1, HLA-DQAT, HLA-DQA2, HLA-DQB1, HLA-DQB2, HLA-DRA, HLA-DRB1, HLA-DRB3, HLA-DRB4, HLA-DRB5, Transporter associated with Antigen Processing 1 (TAP1), Macrophage inhibitory cytokine-1 (MIC-1), MIC-2, CD46, CD59, CD55, CD24, HLA-G, HLA-E, Programmed death-ligand 1 (PD-L1) and/or CD200 gene relative to a wildtype stem cell.

An embodiment of the invention relates to the stem cell above, where the B2M gene is deleted or otherwise inhibited, and where the CD24, HLA-G, HLA-E and/or PD-L1 gene are conditionally overexpressed during certain or predetermined cell differentiation stages.

An embodiment of the invention relates to the stem cell above, where the stem cell is a muscle precursor cell.

An embodiment of the invention relates to the stem cell above, where the expression of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1 and/or MIC-2 gene is reduced relative to a wildtype stem cell.

An embodiment of the invention relates to the stem cell above, where the expression of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1 and/or MIC-2 gene is inhibited.

An embodiment of the invention relates to the stem cell above, where at least one of the HLA-A, HLA-B, HLA-C, B2M, CIITA, CD74, HLA-DMA, HLA-DMB, HLA-DOA, HLA-DOB, HLA-DPA1, HLA-DPB1, HLA-DQAT, HLA-DQA2, HLA-DQB1, HLA-DQB2, HLA-DRA, HLA-DRB1, HLA-DRB3, HLA-DRB4, HLA-DRB5, TAP1, MIC-1 and/or MIC-2 genes is deleted.

An embodiment of the invention relates to the stem cell above, where the PD-L1 gene is conditionally over-expressed relative to a wildtype stem cell.

An embodiment of the invention relates to the stem cell above, where the PD-L1 gene is over-expressed during a first cell-differentiation stage, and where the PD-L1 gene has a reduced level of expression during a second cell-differentiation stage relative to the first cell-differentiation stage.

An embodiment of the invention relates to the stem cell above, where expression of the PD-L1 gene is regulated by a muscle-specific promoter (e.g., a pax7 promotor, a pax3 promotor, a myh4 promotor, a Ankrd1 promotor, a myod1 promotor, a myf5 promotor, or a myog promotor.)

An embodiment of the invention relates to the stem cell above, where the CD24 gene is conditionally over-expressed relative to a wildtype stem cell.

An embodiment of the invention relates to the stem cell above, where the CD24 gene is over-expressed during a first cell-differentiation stage, and where the PD-L1, HLA-G and/or HLA-E gene has a reduced level of expression during a second cell-differentiation stage relative to the first cell-differentiation stage.

An embodiment of the invention relates to the stem cell above, where expression of the CD24 gene is regulated by a pax7 promotor, a pax3 promotor, a myh4 promotor, a Ankrd1 promotor, a myod1 promotor, and myf5 promotor, or a myog promotor.

An embodiment of the invention relates to the stem cell above, where the PD-L1 gene and the CD24 gene are both overexpressed relative to a wildtype stem cell.

An embodiment of the invention relates to the stem cell above, where the PD-L1 gene and the CD24 gene are both regulated by a pax7 promotor, a pax3 promotor, a myh4 promotor, a Ankrd1 promotor, a myod1 promotor, and myf5 promotor, or a myog promotor.

An embodiment of the invention relates to the stem cell above, where the PD-L1 gene and the CD24 gene are inserted into a safe harbor locus of at least one allele of the stem cell.

An embodiment of the invention relates to the stem cell above, where the safe harbor locus includes an AAVS1 locus. Other safe harbor loci include the ROSA26 locus and the CCR5 locus. Other safe harbor loci will be known to persons skilled in the art.

An embodiment of the invention relates to the stem cell above, where the stem cell is one of a pluripotent stem cell or a myogenic stem cell.

An embodiment of the invention relates to the stem cell above, where the stem cell is hypoimmunogenic.

An embodiment of the invention relates to a method of preparing a hypoimmunogenic stem cell, including the steps of: reducing expression of a B2M gene in a stem cell; and conditionally expressing at least one of a PD-L1 gene and a CD24 gene in the stem cell; where the reducing the expression of the B2M gene and conditionally expressing the at least one of the PD-L1 gene and the CD24 gene results in the hypoimmunogenic stem cell.

An embodiment of the invention relates to method above, where reducing expression of the B2M gene includes deletion of a B2M gene from a genomic DNA of the stem cell.

An embodiment of the invention relates to method above, where deletion of the B2M gene from the genomic DNA of the stem cell includes use of an endonuclease.

An embodiment of the invention relates to method above, where conditionally expressing at least one of a PD-L1 gene and a CD24 gene in the stem cell includes inserting at least one of the PD-L1 gene and the CD24 gene into a safe harbor locus of at least one allele of the stem cell.

An embodiment of the invention relates to method above, where the safe harbor locus includes an AAVS1 locus.

An embodiment of the invention relates to method above, where conditionally expressing at least one of a PD-L1 gene and a CD24 gene in the stem cell includes over-expressing at least one of the PD-L1 gene and the CD24 gene during a first cell-differentiation stage, and reducing expression of the at least one of the PD-L1 gene and the CD24 gene during a second cell-differentiation stage relative to the first cell-differentiation stage.

An embodiment of the invention relates to method above, where conditionally expressing at least one of a PD-L1 gene and a CD24 gene in the stem cell includes use of a pax7 promotor, a pax3 promotor, a myh4 promotor, a Ankrd1 promotor, a myod1 promotor, and myf5 promotor, or a myog promotor.

An embodiment of the invention relates to method above, where the PD-L1 gene and the CD24 gene are both conditionally expressed.

An embodiment of the invention relates to a method of generating a myoblast fusion including the steps of: a) engineering or isolating an iPSC having a disrupted HLA and/or BLA gene and a PD-L1 and/or CD24 gene under the control of a conditional promoter; b) differentiating the iPSC into a satellite cell capable of evading the immune system, wherein the satellite cell has a reduced expression of HLA and/or BLA and an increased expression of PD-L1 and/or CD24; differentiating the satellite cell into a myoblast; and allowing the myoblast to fuse with other myoblasts such that a myoblast fusion is formed, wherein the myoblast in the myoblast fusion has a reduced expression of PD-L1 and/or CD24 as compared to the satellite cell.

In certain embodiments, the stem cell is a myogenic stem cell having modulated expression of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1, and/or MIC-2; b) over-expressed CD46, CD59, CD55 and c) a PD-L1, HLA-G, HLA-E and/or CD24 gene relative to a wildtype myogenic stem cell.

An embodiment relates to the myogenic stem cell above having modulated expression of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1, and/or MIC-2; b) over-expressed CD46, CD59, CD55 and c) a PD-L1, HLA-G, HLA-E and/or CD24 gene relative to a wildtype myogenic stem cell.

An embodiment relates to the myogenic stem cell above where the expression of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1, and/or MIC-2 genes are reduced relative to a wildtype stem cell.

An embodiment relates to the myogenic stem cell above where the expression of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1, and/or MIC-2 genes are inhibited.

An embodiment relates to the myogenic stem cell above where at least one of the HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1, and/or MIC-2 genes are deleted.

An embodiment relates to the myogenic stem cell above where the PD-L1, HLA-G, HLA-E and/or CD24 genes are conditionally over-expressed relative to a wildtype stem cell.

An embodiment relates to the myogenic stem cell above where the PD-L1, HLA-G, HLA-E and/or CD24 genes are over-expressed during a first cell-differentiation stage, and wherein the PD-L1 gene has a reduced level of expression during a second cell-differentiation stage relative to the first cell-differentiation stage.

An embodiment relates to the myogenic stem cell above where expression of the PD-L1, HLA-G, HLA-E and/or CD24 genes are regulated by a pax7 promotor, a pax3 promotor, a myh4 promotor, a Ankrd1 promotor, a myod1 promotor, and myf5 promotor, or a myog promotor.

An embodiment relates to the myogenic stem cell above where the CD24 gene is conditionally over-expressed relative to a wildtype stem cell.

An embodiment relates to the myogenic stem cell above where the CD24 gene is over-expressed during a first cell-differentiation stage, and wherein the PD-L1 gene has a reduced level of expression during a second cell-differentiation stage relative to the first cell-differentiation stage.

An embodiment relates to the myogenic stem cell above where expression of the CD24 gene is regulated by a pax7 promotor, a pax3 promotor, a myh4 promotor, a Ankrd1 promotor, a myod1 promotor, and myf5 promotor, or a myog promotor.

An embodiment relates to the myogenic stem cell above where the PD-L1 gene and the CD24 gene are both overexpressed relative to a wildtype stem cell.

An embodiment relates to the myogenic stem cell above where the PD-L1 gene and the CD24 gene are both regulated by a pax7 promotor, a pax3 promotor, a myh4 promotor, a Ankrd1 promotor, a myod1 promotor, and myf5 promotor, or a myog promotor.

An embodiment relates to the myogenic stem cell above where the PD-L1 gene and the CD24 gene are inserted into a safe harbor locus of at least one allele of the stem cell.

An embodiment relates to the myogenic stem cell above where the safe harbor locus comprises an AAVS1 locus.

An embodiment relates to the myogenic stem cell above where the myogenic stem cell is a satellite cell.

An embodiment relates to the myogenic stem cell above where the myogenic stem cell is hypoimmunogenic.

An embodiment of the invention relates to a method of preparing a hypoimmunogenic stem cell, including the steps of: reducing expression of a B2M gene in a stem cell; and conditionally expressing at least one of a PD-L1 gene and a CD24 gene in the stem cell; where the reducing the expression of the B2M gene and conditionally expressing the at least one of the PD-L1 gene and the CD24 gene results in the hypoimmunogenic stem cell.

An embodiment relates to the method above, where reducing expression of the B2M gene comprises deletion of a B2M gene from a genomic DNA of the stem cell.

An embodiment relates to the method above, where deletion of the B2M gene from the genomic DNA of the stem cell comprises use of an endonuclease.

An embodiment relates to the method above, where conditionally expressing at least one of a PD-L1 gene and a CD24 gene in the stem cell comprises inserting at least one of the PD-L1 gene and the CD24 gene into a safe harbor locus of at least one allele of the stem cell.

An embodiment relates to the method above, where the safe harbor locus comprises an AAVS1 locus.

An embodiment relates to the method above, where conditionally expressing at least one of a PD-L1 gene and a CD24 gene in the stem cell comprises over-expressing at least one of the PD-L1 gene and the CD24 gene during a first cell-differentiation stage, and reducing expression of the at least one of the PD-L1 gene and the CD24 gene during a second cell-differentiation stage relative to the first cell-differentiation stage.

An embodiment relates to the method above, where conditionally expressing at least one of a PD-L1 gene and a CD24 gene in the stem cell comprises use of a pax7 promotor, a pax3 promotor, a myh4 promotor, a Ankrd1 promotor, a myod1 promotor, and myf5 promotor, or a myog promotor.

An embodiment relates to the method above, where the PD-L1 gene and the CD24 gene are both conditionally expressed.

In certain embodiments, the stem cell is an induced Pluripotent Stem Cell (iPSC). In some embodiments, the stem cell is a myogenic stem cell. In some embodiments, the stem cell has a) a disrupted HLA and/or BLA gene; and b) a PD-L1 and/or a CD47 gene under the control of a conditional promoter. In some such embodiments, the conditional promotor is the Pax-7 promoter, but it can be any promoter that is active only during certain stages of cell differentiation. More specifically, the promoter is active during a satellite cell stage and during a myoblast cell stage, but it turns off once fused myoblasts differentiate into muscle fiber.

An embodiment of the invention relates to a stem cell having modulated expression of a β-Microglobulin (B2M) gene, a PD-L1 gene, and a CD47 gene relative to a wildtype stem cell.

An embodiment of the invention relates to the stem cell above, where the B2M gene is deleted or otherwise inhibited, and where the PD-L1 gene and the CD47 gene are conditionally overexpressed during certain or predetermined cell differentiation stages.

An embodiment of the invention relates to the stem cell above, where the stem cell is a muscle precursor cell.

An embodiment of the invention relates to the stem cell above, where the expression of the B2M gene is reduced relative to a wildtype stem cell.

An embodiment of the invention relates to the stem cell above, where the expression of the B2M gene is inhibited.

An embodiment of the invention relates to the stem cell above, where at least one B2M gene is deleted.

An embodiment of the invention relates to the stem cell above, where the PD-L1 gene is conditionally over-expressed relative to a wildtype stem cell.

An embodiment of the invention relates to the stem cell above, where the PD-L1 gene is over-expressed during a first cell-differentiation stage, and where the PD-L1 gene has a reduced level of expression during a second cell-differentiation stage relative to the first cell-differentiation stage.

An embodiment of the invention relates to the stem cell above, where expression of the PD-L1 gene is regulated by a pax7 promotor, a pax3 promotor, a myh4 promotor, a Ankrd1 promotor, a myod1 promotor, and myf5 promotor, or a myog promotor.

An embodiment of the invention relates to the stem cell above, where the CD47 gene is conditionally over-expressed relative to a wildtype stem cell.

An embodiment of the invention relates to the stem cell above, where the CD47 gene is over-expressed during a first cell-differentiation stage, and where the PD-L1 gene has a reduced level of expression during a second cell-differentiation stage relative to the first cell-differentiation stage.

An embodiment of the invention relates to the stem cell above, where expression of the CD47 gene is regulated by a pax7 promotor, a pax3 promotor, a myh4 promotor, a Ankrd1 promotor, a myod1 promotor, and myf5 promotor, or a myog promotor.

An embodiment of the invention relates to the stem cell above, where the PD-L1 gene and the CD47 gene are both overexpressed relative to a wildtype stem cell.

An embodiment of the invention relates to the stem cell above, where the PD-L1 gene and the CD47 gene are both regulated by a pax7 promotor, a pax3 promotor, a myh4 promotor, a Ankrd1 promotor, a myod1 promotor, and myf5 promotor, or a myog promotor.

An embodiment of the invention relates to the stem cell above, where the PD-L1 gene and the CD47 gene are inserted into a safe harbor locus of at least one allele of the stem cell.

An embodiment of the invention relates to the stem cell above, where the safe harbor locus includes an AAVS1 locus.

An embodiment of the invention relates to the stem cell above, where the stem cell is a pluripotent stem cell.

An embodiment of the invention relates to the stem cell above, where the stem cell is hypoimmunogenic.

An embodiment of the invention relates to a method of preparing a hypoimmunogenic stem cell, including the steps of: reducing expression of a B2M gene in a stem cell; and conditionally expressing at least one of a PD-L1 gene and a CD47 gene in the stem cell; where the reducing the expression of the B2M gene and conditionally expressing the at least one of the PD-L1 gene and the CD47 gene results in the hypoimmunogenic stem cell.

An embodiment of the invention relates to method above, where reducing expression of the B2M gene includes deletion of a B2M gene from a genomic DNA of the stem cell.

An embodiment of the invention relates to method above, where deletion of the B2M gene from the genomic DNA of the stem cell includes use of an endonuclease.

An embodiment of the invention relates to method above, where conditionally expressing at least one of a PD-L1 gene and a CD47 gene in the stem cell includes inserting at least one of the PD-L1 gene and the CD47 gene into a safe harbor locus of at least one allele of the stem cell.

An embodiment of the invention relates to method above, where the safe harbor locus includes an AAVS1 locus. Other safe harbor loci include the ROSA26 locus and the CCR5 locus. Other safe harbor loci will be known to persons skilled in the art.

An embodiment of the invention relates to method above, where conditionally expressing at least one of a PD-L1 gene and a CD47 gene in the stem cell includes over-expressing at least one of the PD-L1 gene and the CD47 gene during a first cell-differentiation stage, and reducing expression of the at least one of the PD-L1 gene and the CD47 gene during a second cell-differentiation stage relative to the first cell-differentiation stage.

An embodiment of the invention relates to method above, where conditionally expressing at least one of a PD-L1 gene and a CD47 gene in the stem cell includes use of a pax7 promotor, a pax3 promotor, a myh4 promotor, a Ankrd1 promotor, a myod1 promotor, and myf5 promotor, or a myog promotor.

An embodiment of the invention relates to method above, where the PD-L1 gene and the CD47 gene are both conditionally expressed.

An embodiment of the invention relates to a method of generating a myoblast fusion including the steps of: a) engineering or isolating an iPSC having a disrupted HLA and/or BLA gene and a PD-L1 and/or CD47 gene under the control of a conditional promoter; b) differentiating the iPSC into a satellite cell capable of evading the immune system, wherein the satellite cell has a reduced expression of HLA and/or BLA and an increased expression of PD-L1 and/or CD47; differentiating the satellite cell into a myoblast; and allowing the myoblast to fuse with other myoblasts such that a myoblast fusion is formed, wherein the myoblast in the myoblast fusion has a reduced expression of PD-L1 and/or CD47 as compared to the satellite cell.

Some embodiments of the invention include methods, compositions and kits for producing populations of myogenic progenitor cells. Also included are methods and compositions for treating or preventing a muscle disease or disorder in a subject (e.g., Duchenne muscular dystrophy (DMD)).

Provided herein is a novel approach to generating human PAX7::GFP+ cells that can be survive as quiescent and functional local skeletal muscle stem cells in niche area of in vivo environments.

Also provided, is that these cells could be maintained and expanded ex vivo. When transplanted in vivo, they could participate in the muscle regeneration by fusing into muscle fiber as well as becoming mononucleated PAX7 expressing cells residing under basal lamina.

Myogenesis is the formation of muscular tissue, e.g., particularly during embryonic development. Muscle fibers generally form the fusion of myoblasts into multinucleated fibers called myotubes. In the early development of an embryo, myoblasts can either proliferate, or differentiate into a myotube. What controls this choice in vivo is generally unclear. If placed in cell culture, most myoblasts will proliferate if enough fibroblast growth factor (FGF) or another growth factor is present in the medium surrounding the cells. When the growth factor runs out, the myoblasts cease division and undergo terminal differentiation into myotubes. Myoblast differentiation proceeds in stages. The first stage, involves cell cycle exit and the commencement of expression of certain genes. The second stage of differentiation involves the alignment of the myoblasts with one another. Studies have shown that even rat and chick myoblasts can recognize and align with one another, suggesting evolutionary conservation of the mechanisms involved. The third stage is the actual cell fusion itself. In this stage, the presence of calcium ions is critical. In mice, fusion is aided by a set of metalloproteinases called meltrins and a variety of other proteins still under investigation. Fusion involves recruitment of actin to the plasma membrane, followed by close apposition and creation of a pore that subsequently rapidly widens.

During embryogenesis, the dermomyotome and/or myotome in the somites contain the myogenic progenitor cells that will evolve into the prospective skeletal muscle. The determination of dermomyotome and myotome is regulated by a gene regulatory network that includes a member of the T-box family, tbx6, ripply1, and mesp-ba. Skeletal myogenesis depends on the strict regulation of various gene subsets in order to differentiate the myogenic progenitors into myofibers. Myogenic progenitors expressed the transcription factors pax3 and pax7. Basic helix-loop-helix (bHLH) transcription factors, MyoD, Myf5, myogenin, and MRF4 are critical to its formation. MyoD and Myf5 enable the differentiation of myogenic progenitors into myoblasts, followed by myogenin, which differentiates the myoblast into myotubes. MRF4 is important for blocking the transcription of muscle-specific promoters, enabling skeletal muscle progenitors to grow and proliferate before differentiating.

Myogenic Progenitor Cells

Skeletal muscles of adult mammalian species exhibit a capacity to adapt to physiological demands such as growth, training, and injury. The processes by which these adaptations occur are attributed to a small population of mononuclear cells that is resident in adult skeletal muscle and has been referred to as satellite cells. Satellite cells are tissue specific myogenic stem cells, which can self-renew and give rise to muscle precursor known as myoblasts, which serve the ‘building blocks’ of skeletal muscle. Skeletal muscle fibers are terminally differentiated and the nuclei in these multinucleated cells are incapable of DNA synthesis or mitotic division. Increases in muscle fiber numbers or in numbers of muscle fiber nuclei are due to proliferation and subsequent differentiation of myoblasts. In adult skeletal muscle, satellite cells remain in a mitotically quiescent state, which can, upon muscle injury, re-enter the cell cycle, undergo asymmetric cell division to give rise to a daughter satellite cell and a transient amplifying myoblast that can undergo several rounds of divisions. Upon differentiation, myoblasts fuse with one another or with preexisting muscle fibers, and also commence expression of a set of muscle-specific myofibrillary and contractile proteins. Quiescent myogenic satellite cells are physically distinct from the adult myofibers as they reside in indentations between the sarcolemma and the basal lamina. In the case of muscle injury, some of these cells will remain as progenitor cells whereas others will differentiate into new muscle fibers. In response to stimuli such as myotrauma, myogenic progenitor cells become activated, proliferate, and express myogenic markers. Ultimately, these cells fuse to existing muscle fibers or fuse together to form new myofibers during regeneration of damaged skeletal muscle.

Some embodiments of the invention include methods of producing a population of myogenic progenitor cells (MPCs). In some embodiments, the method comprises obtaining a cell population from a subject (e.g., a skeletal cell population), producing a pluripotent stem cell (PSC) population from the cell population, wherein the PSC population is an embryonic stem cell (ESC) population, and producing the MPC population from the PSC population.

In aspects, the pluripotent cell is an embryonic stem (ES) cell (or ESC). In one embodiment, the pluripotent cell is a non-human ES cell. In one embodiment, the pluripotent cell is an induced pluripotent stem (iPS) cell (or iPSC). In one embodiment, the iPS cell is derived from a fibroblast. In one embodiment, the iPSC is derived from a human blood cell. In one embodiment, the pluripotent cell is a hematopoietic stem cell (HSC). In one embodiment, the pluripotent cell is a neuronal stem cell (NSC). In one embodiment, the pluripotent cell is an epiblast stem cell. In one embodiment, the pluripotent cell is a tissue specific stem cell or developmentally restricted progenitor cell. In one embodiment, the pluripotent cell is a rodent pluripotent cell. In one embodiment, the rodent pluripotent cell is a rat pluripotent cell. In one embodiment, the rat pluripotent cell is a rat ES cell. In one embodiment, the rodent pluripotent cell is a mouse pluripotent cell. In one embodiment, the pluripotent cell is a mouse embryonic stem (ES) cell.

In aspects, the PSC population is cultured in a cell culture medium comprising basic fibroblast growth factor (FGF2) and/or fibroblast growth factor 8.

In additional aspects, the MPCs express myogenic markers, wherein the myogenic markers may include paired box protein (PAX7) or MyoD. Additionally, the MPCs express green fluorescent protein (GFP).

The methods for producing an MPC population is effective to increase PAX7 expression in cells of the MPC population. For example, the expression may be increased by at least 0, 20, 30, 40, 50, 60, 70, 80, 90, 100, 125, 150, 175, or 200% compared to a reference. In embodiments, PAX7 expression in the myogenic progenitor cell population increases by at least 100%. In embodiments, the level of PAX7 expression in the myogenic progenitor cell population increases by at least 125%. In embodiments, the level of PAX7 expression in the myogenic progenitor cell population increases by at least 150%. In embodiments, the level of PAX7 expression in the myogenic progenitor cell population increases by at least 50%, 100%, 200%, 300%, 400%, 500%, 600%, 700%, 800%, 900%, 1000%, 1100%, or 1200%. In embodiments, the level of PAX7 expression in the myogenic progenitor cell population increases by about 50% to about 1200%.

An amino acid sequence for human PAX7 is publicly available in the NCBI GenBank database under accession number NP_002575.1

A nucleotide sequence that encodes human PAX7 is publicly available in the GenBank database under accession number NM_002584.2

An amino acid sequence for mouse PAX7 is publicly available in the NCBI GenBank database under accession number NP_035169.1

A nucleotide sequence that encodes mouse PAX7 is publicly available in the GenBank database under accession number NM_011039.2

The methods for producing an MPC population is effective to increase MyoD expression in cells of the MPC population. For example, the expression may be increased by at least 0, 20, 30, 40, 50, 60, 70, 80, 90, 100, 125, 150, 175, or 200% compared to a reference. In embodiments, MyoD expression in the myogenic progenitor cell population increases by at least 100%. In embodiments, the level of MyoD expression in the myogenic progenitor cell population increases by at least 125%. In embodiments, the level of MyoD expression in the myogenic progenitor cell population increases by at least 150%. In embodiments, the level of MyoD expression in the myogenic progenitor cell population increases by at least 50%, 100%, 200%, 300%, 400%, 500%, 600%, 700%, 800%, 900%, 1000%, 1100%, or 1200%. In embodiments, the level of MyoD expression in the myogenic progenitor cell population increases by about 50% to about 1200%.

An amino acid sequence for human MyoD is publicly available in the NCBI GenBank database under accession number NP_002469.2

A nucleotide sequence that encodes human MyoD is publicly available in the GenBank database under accession number NM_002478.5

An amino acid sequence for mouse MyoD is publicly available in the NCBI GenBank database under accession number NP_034996.2

A nucleotide sequence that encodes mouse MyoD is publicly available in the GenBank database under accession number NM_010866.2

In other aspects, the methods for producing an MPC population include that the cell population is cultured and expanded ex vivo for at least 30 days. In other aspects, the cell population is cultured and expanded ex vivo for at least 5 days, at least 10 days, at least 20 days, at least 30 days, at least 40 days, at least 50 days.

In aspects, the cell population is cultured to expand their number. For example, the number may be increased by at least 0, 20, 30, 40, 50, 60, 70, 80, 90, 100, 125, 150, 175, or 200%. In embodiments, the number may increase by at least 50%, 100%, 200%, 300%, 400%, 500%, 600%, 700%, 800%, 900%, 1000%, 1100%, or 1200%. In embodiments, the cell population is cultured and expanded by at least 10%, by at least 20%, by at least 30%, by at least 40%, by at least 50%.

In additional examples, the MPC population is produced from a population of cells obtained from the subject, e.g., skeletal cells. In other examples, the PSC population is an iPS cell population.

Also provided herein is a population of myogenic progenitor cells produced according to the methods described herein.

Some embodiments of the invention include a method of preventing or treating muscle diseases or disorders in a subject in need thereof. In further embodiments, the method comprises administering to the subject an effective amount of the population of myogenic progenitor cells produced according to the methods described herein. For example, methods for preventing or treating a muscle disease or disorder include administering a producing a population of myogenic progenitor cells (MPCs), wherein a cell population is obtained from a subject (e.g., a skeletal cell population), producing a pluripotent stem cell (PSC) population from the cell population, wherein the PSC population is an embryonic stem cell (ESC) population, and producing the MPC population from the PSC population.

In embodiments, the muscle disease or disorder comprises a degenerative muscular wasting disorders or volumetric muscle loss. In some embodiments, the muscle disease is a muscular dystrophy or a muscle wasting disease. Exemplary muscular dystrophies include Duchenne Muscular Dystrophy, Becker Muscular Dystrophy, Limb Girdle Muscular Dystrophy, Facioscapulohumeral Muscular Dystrophy, Oculopharyngeal muscular dystrophy, Emery-Dreifuss muscular dystrophy, Fukuyama-type congenital muscular dystrophy, Miyoshi myopathy, Ullrich congenital muscular dystrophy, Steinert Muscular Dystrophy.

In embodiments, the muscular dystrophy comprises Duchenne muscular dystrophy (DMD).

In aspects, the cells are isolated before the muscle disease (e.g., Duchenne muscular dystrophy) begins in the subject. In other aspects, the cells are isolated after the muscle disease (e.g., Duchenne muscular dystrophy) beings in the subject.

In aspects, the cells are cultured to expand their number before being administered to the subject. In aspects, the cell population is cultured to expand their number. For example, the number may be increased by at least 0, 20, 30, 40, 50, 60, 70, 80, 90, 100, 125, 150, 175, or 200%. In embodiments, the number may increase by at least 50%, 100%, 200%, 300%, 400%, 500%, 600%, 700%, 800%, 900%, 1000%, 1100%, or 1200%. In embodiments, the cell population is cultured and expanded by at least 10%, by at least 20%, by at least 30%, by at least 40%, by at least 50%.

In embodiments, provided herein are methods for increasing the level of an early myogenic marker in a subject in need thereof. In further embodiments, the method comprises administering to the subject, an effective amount of the population of myogenic progenitor cells produced according to the methods described herein.

In other embodiments, the methods for treating a muscle disease or disorder (e.g., Duchenne muscular dystrophy) comprise administering to a subject a population of myogenic progenitor cells produced according to the methods described herein, in combination with methods for controlling the outset of symptoms. In particular, the combination treatment can include administering corticosteroids (e.g., such as prednisolone and deflazacort), β2 agonists (e.g., salbutamol (e.g., albuterol). Additionally, combination therapy may include nonjarring physical activity (e.g., including, swimming), physical therapy, orthopedic appliances (such as braces and wheelchairs), appropriate respiratory support.

Some embodiments of the invention include a kit for producing a myogenic progenitor cells is provided. In embodiments, the kit comprises a cell culture media or a cell culture medium wherein the cell culture medium is suitable for culturing a pluripotent stem cell (PSC) population or a myogenic stem cell population.

Various alternative reagents (e.g., coatings, disassociation agents, stimulation reagents, differentiation reagents, and culture reagents such as media) may be used in embodiments herein. No specific set of reagents is required for the culturing of, e.g., PSCs, ES cells, and iPSCs. However, non-limiting examples are provided below.

In embodiments, the kit comprises a medium for growth and expansion of human iPS and hES cells.

In embodiments, the kit comprises a reagent for ES and/or iPS cell selection and/or passaging. In embodiments, the reagent is ReLeSR™ passaging reagent (Stemcell Technologies, Vancouver Canada, Catalog No. 05872 or 05873). In embodiments, the reagent is mTeSR™1 (Stemcell Technologies, Vancouver Canada, Catalog No. 85850 or 85857). In embodiments, the reagent is Vitronectin XF™ (Stemcell Technologies, Vancouver Canada, Catalog No. 07180 or 07190). In embodiments, the reagent is Gentle Cell Dissociation Reagent (Stemcell Technologies, Vancouver Canada, Catalog No. 07174). Many plate coating reagents and ES/iPSC medium are commercially available and will be known to those skilled in the art. In embodiments, any plate coating reagent may be used. In embodiments, the coating reagent is Matrigel from Corning (New York N.Y., USA).

In embodiments, the kit comprises a reagent comprising one or more cell-dissociation enzymes. In embodiments, the reagent is TrypLE™ cell dissociation reagent (ThermoFisher Catalog No: A1285901). In embodiments, the reagent is TrypLE™ Express (Thermo Fisher SKU No. 12604-013). In embodiments, the reagent is StemPro™ Accutase™ Cell Dissociation Reagent (Thermo Fisher Catalog No. A1110501). Various disassociation reagents are known in the art and may be used. In embodiments, cells may be physically scraped off a culture surface (such as a plate).

In embodiments, the kit comprises basic fibroblast growth factor (FGF2) and/or fibroblast growth factor 8.

In embodiments, a cell culture medium in the kit is suitable for culturing a PSC population.

In embodiments, a cell culture medium in the kit comprises about 5%, 4.5%, 4%, 3.5%, 3%, 2.5%, 2%, 1.5%, 1%, or 0.5% serum. In embodiments, the kit is configured for use of about 5%, 4.5%, 4%, 3.5%, 3%, 2.5%, 2%, 1.5%, 1%, or 0.5% serum or less.

In embodiments, a cell culture medium in the kit is a serum-free cell culture medium.

In embodiments, the kit does not comprise serum.

As described above, two major barriers for cell therapy based on stem cells are the risk of rejection by the recipient's immune system and/or the risk of tumor formation.

To [symbol] overcome this, the present invention provides described an immune-evading [symbol] strategy to generate hypoimmunogenic satellite cells. An exemplary method to generate hypoimmunogenic satellite cells includes: Using the CrispR/cas9 system in the parental stem cell donor line to inactivate expression of HLA-A, HLA-B, HLA-C, B2M, CIITA, CD74, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, TAP1, MIC-1, and/or MIC-2, which are a critical components of the major [symbol] histocompatibility complex (MHC) class I and II. Cells lacking MHC class I are recognized and targeted by natural killer (NK) cells. NK cells are innate lymphocytes that will kill cells that down-regulate MHC class I (MHC-I) through “missing-self” recognition by releasing cytotoxic molecules. However, NK cells express the receptors NKG2D and Killer cell Immunoglobulin-like Receptors (KIRs) that if activated will either activate or inhibit NK cell activity and release of cytotoxic molecules, respectively. MIR-1 and MIR-2 interact with NKG2D to activate NK cells' cytotoxic release, whereas HLA-G, HLA-E and PD-L1 interact with KIRs, which blocks the release of cytotoxic molecules. Therefore, a strategy where satellite cells derived from the parental donor line absent of MIC-1 and MIC-2 and express HLA-G, HLA-E and/or PD-L1 on the surface has been employed, which upon interaction between hypoimmunogenic cells deprived of MIC-1 and MIC-2 and expressing HLA-G, HLA-E and/or PD-L1 will prevent cytotoxic release from NK cells.

Another process that can negatively affect engraftment of foreign cells is phagocytosis mediated by macrophages. Macrophages express SIRPα on the surface, which serves as an inhibitory receptor for macrophage phagocytosis. SIRPα is activated by interaction with the “don't-eat me” surface molecule CD24. Therefore, an additional strategy was designed where satellite cells derived from the parental donor line express CD24 on the surface. To avoid random integration and positional effects on transgene expression, PD-L1 and CD24 are knocked-into the AAVS1 safe harbor locus. The pax7:myod1 fusion promoter has been inserted in front of PD-L1 and CD24 to exclusively express these factors in satellite cells. Thus, if satellite cells differentiate to other non-muscle lineages than the skeletal muscle, these cells will downregulate expression of CD24 and PD-L1 due to pax7:myod1 inactivation, and be prone to recognition by NK cells and macrophages.

To avoid adaptive immune recognition by T-Cells, B2M is deleted; to protect cells from NK cells, CD24 overexpression is driven by pax7:myod1 fusion promoter; and to inhibit macrophage engulfment, CD24 overexpression is driven by pax7:myod1 fusion promoter.

An amino acid sequence for human HLA-G is publicly available in the NCBI GenBank database under accession number NP_001350496.

A nucleotide sequence that encodes human HLA-G is publicly available in the GenBank database under accession number NM_001363567.

An amino acid sequence for human CD24 is publicly available in the NCBI GenBank database under accession number NP_037362.

A nucleotide sequence that encodes human CD24 is publicly available in the GenBank database under accession number NM_013230.

An amino acid sequence for human HLA-E is publicly available in the NCBI GenBank database under accession number NP_005507.

A nucleotide sequence that encodes human HLA-E is publicly available in the GenBank database under accession number NM_005516.

An amino acid sequence for human PD-L1 is publicly available in the NCBI GenBank database under accession number NP_001156884.

A nucleotide sequence that encodes human PD-L1 is publicly available in the GenBank database under accession number NM_001163412.

An amino acid sequence for human CD200 is publicly available in the NCBI GenBank database under accession number NP_005935.4.

A nucleotide sequence that encodes human CD200 is publicly available in the GenBank database under accession number NM_005944.7.

An amino acid sequence for human CD46 is publicly available in the NCBI GenBank database under accession number NP_002380.3.

A nucleotide sequence that encodes human CD46 is publicly available in the GenBank database under accession number NM_002389.4.

An amino acid sequence for human CD55 is publicly available in the NCBI GenBank database under accession number NP_000565.1.

A nucleotide sequence that encodes human CD55 is publicly available in the GenBank database under accession number NM_000574.5.

An amino acid sequence for human CD59 is publicly available in the NCBI GenBank database under accession number NP_976075.1.

A nucleotide sequence that encodes human is publicly available in the GenBank database under accession number NM_203330.2.

### Example

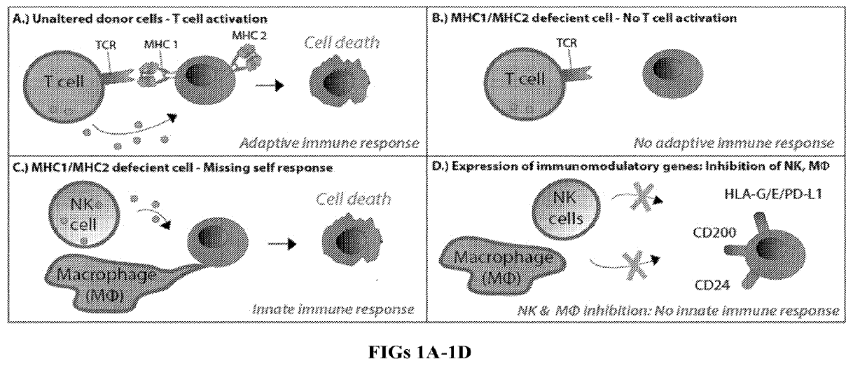

An example embodiment is provided in the schematics depicted in FIGS. 1A-1D, 2, and 3A-3C.

FIGS. 1A-1D depict a general schematic showing how immune evasion is achieved according to an embodiment of the invention. FIG. 1A shows that normal unaltered cells expresses MHC1 and potentially MHC2, which both can be recognized by T cells as part of the adaptive immune response leading to T cell mediated destruction of the cells. FIG. 1B shows that T cells are unable to recognize cells when MHC1 and MHC2 is deleted. FIG. 1C shows that absence of MHC1 and MHC2 lead to ‘missing self’ recognition by Natural Killer (NK) cells along with Macrophage-based phagocytosis as part of the innate immune response. FIG. 1D shows that expression of the immunomodulatory factors CD200, CD24, PD-L1, and HLA-G/E inhibits NK cells and Macrophages, thus diminishing activation of innate immunity resulting in immune tolerance.

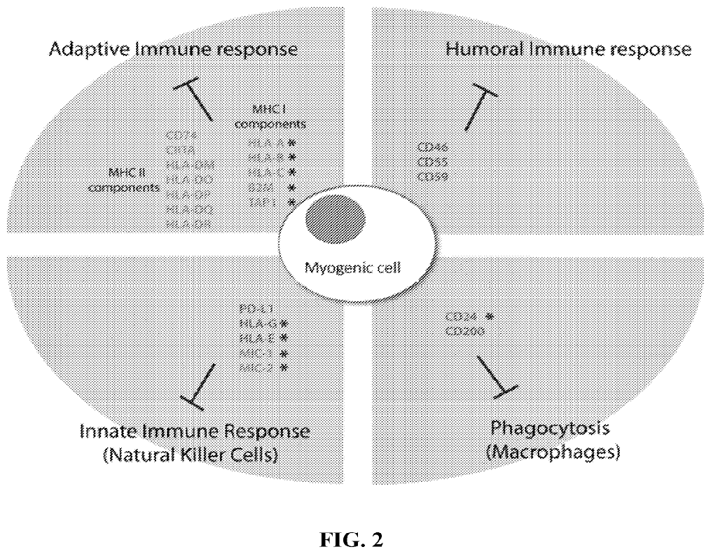

FIG. 2 is a schematic showing a gene alteration strategy and role during immunity according to an embodiment of the invention. In FIG. 2, one or more of genes CD74, CIITA, HLA-DM, HLA-DO, HLA-DP, HLA-DQ, HLA-DR, HLA-A, HLA-B, HLA-CB2M, TAP1, MIC-1, and MIC-2 are deleted/inhibited; one or more of genes CD46, CD55, and CD59 are overexpressed; and one or more of genes PD-L1, HLA-G, HLA-E, CD24, and CD200 are conditionally over-expressed via tissue specific promoter. Genes marked by an asterisk* are genes essential for cloaking (i.e. immune evasion) in some embodiments.

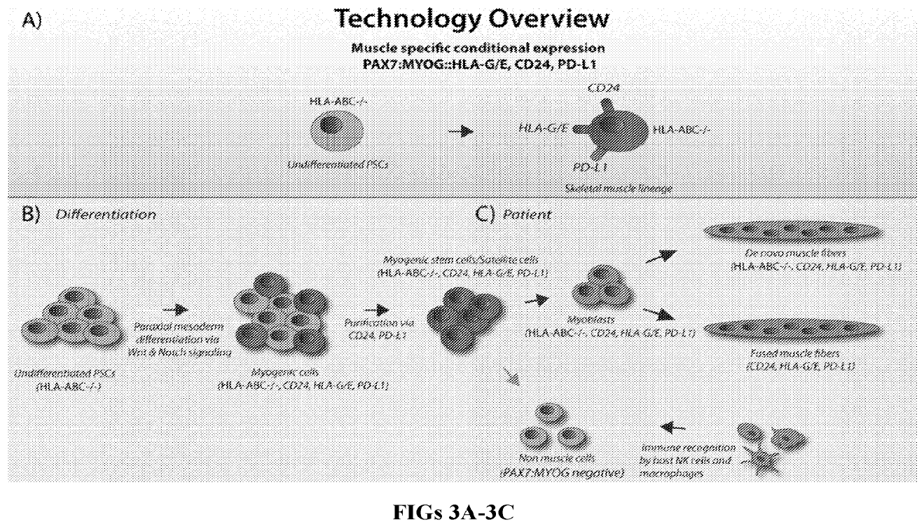

FIGS. 3A-3C show the muscle-specific conditional expression of immunomodulatory factors according to an embodiment of the invention. In FIG. 3A, the muscle specific fusion promoter (PAX7:MYOG) drives expression of specific immunomodulatory genes HLA-G/E, CD24 and PD-L1. In FIG. 3B, myogenic cells differentiated from myogenic stem cells express CD24, HLA-G/E and PD-L1 and are purified via conditional expression of CD24, HLA-G/E and/or PD-L1. In FIG. 3C, myoblasts fuse to generate new (de novo) muscle fibers or fuse with the host's existing muscle fibers. Non-muscle cells will not express muscle specific promoters and thus the immunomodulatory factors HLA-G/E, CD24 and PD-L1 allowing the host' innate immune system (NK cells and macrophages) to recognize and target the cells for destruction.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described. Additional methods of generating human stem cells are disclosed in US Patent Application Publication No. 2018/0360887, which is hereby incorporated by reference in its entirety.

1. A myogenic stem cell comprising a modulated expression of a HLA-A gene, a HLA-B gene, a HLA-C gene, a β-Microglobulin (B2M) gene, a CIITA gene, a CD74 gene, a TAP1 gene, a MIC-1 gene, a MIC-2 gene, a CD24 gene, an HLA-G gene, and an HLA-E gene relative to a wildtype stem cell.

2. The myogenic stem cell of claim 1, wherein the modulated expression of at least one of said HLA-A gene, said HLA-B gene, said HLA-C gene, said β-Microglobulin (B2M) gene, said CIITA gene, said CD74 gene, said TAP1 gene, said MIC-1 gene, and said MIC-2 gene is reduced relative to a wildtype stem cell.

3. The myogenic stem cell of claim 1, wherein an expression of said HLA-A gene, said HLA-B gene, said HLA-C gene, said β-Microglobulin (B2M) gene, said CIITA gene, said CD74 gene, said TAP1 gene, and said MIC-1 gene is inhibited.

4. The myogenic stem cell of claim 1, wherein said HLA-A gene, said HLA-B gene, said HLA-C gene, said β-Microglobulin (B2M) gene, said CIITA gene, said CD74 gene, said TAP1 gene, said MIC-1 gene, and said MIC-2 gene are deleted.

5. The myogenic stem cell of claim 1, wherein at least one of the CD24 gene, the HLA-G gene, and the HLA-E gene is conditionally over-expressed relative to a wildtype stem cell.

6. The myogenic stem cell of claim 5, wherein at least one of the CD24 gene, the HLA-G gene, and the HLA-E gene is over-expressed during a first cell-differentiation stage, and wherein the at least one of the CD24 gene, the HLA-G gene, and the HLA-E gene has a reduced level of expression during a second cell-differentiation stage relative to the first cell-differentiation stage.

7. The myogenic stem cell of claim 5, wherein expression of at least one of the CD24 gene, the HLA-G gene, and the HLA-E gene is regulated by a pax7 promotor, a pax3 promotor, a myh4 promotor, a Ankrd1 promotor, a myod1 promotor, and myf5 promotor, or a myog promotor.

8. The myogenic stem cell of claim 1, wherein the CD24 gene, the HLA-G gene, and the HLA-E gene are conditionally over-expressed relative to a wildtype stem cell.

9. The myogenic stem cell of claim 8, wherein the CD24 gene, the HLA-G gene, and the HLA-E gene are over-expressed during a first cell-differentiation stage, and wherein the CD24 gene, the HLA-G gene, and the HLA-E gene have a reduced level of expression during a second cell-differentiation stage relative to the first cell-differentiation stage.

10. The myogenic stem cell of claim 8, wherein expression of the CD24 gene, the HLA-G gene, and the HLA-E gene are regulated by a pax7 promotor, a pax3 promotor, a myh4 promotor, a Ankrd1 promotor, a myod1 promotor, and myf5 promotor, or a myog promotor.

11. The myogenic stem cell of claim 1, further comprising an increased expression of at least one of a CD46 gene, a CD59 gene, and a CD55 gene relative to a wildtype stem cell.

12. The myogenic stem cell of claim 11, comprising an increased expression of each of the CD46 gene, the CD59 gene, and the CD55 gene relative to a wildtype stem cell.

13. The myogenic stem cell of claim 1, wherein at least one of the CD24 gene, the HLA-G gene, and the HLA-E gene is inserted into a safe harbor locus of at least one allele of the stem cell.

14. The myogenic stem cell of claim 13, wherein the safe harbor locus comprises an AAVS1 locus.

15. The myogenic stem cell of claim 1, wherein the myogenic stem cell is a satellite cell.

16. The myogenic stem cell of claim 1, wherein the stem cell is hypoimmunogenic.

17. The myogenic stem cell of claim 1, further comprising a reduced expression of at least one of an HLA-DMA gene, an HLA-DMB gene, an HLA-DOA gene, an HLA-DOB gene, an HLA-DPA1 gene, an HLA-DPB1 gene, an HLA-DQA1 gene, an HLA-DQA2 gene, an HLA-DQB1 gene, an HLA-DQB2 gene, an HLA-DRA gene, an HLA-DRB1 gene, an HLA-DRB3 gene, an HLA-DRB4 gene, and an HLA-DRB5 gene relative to a wildtype stem cell.

18. The myogenic stem cell of claim 17, wherein at least one of the HLA-DMA gene, the HLA-DMB gene, the HLA-DOA gene, the HLA-DOB gene, the HLA-DPA1 gene, the HLA-DPB1 gene, the HLA-DQA1 gene, the HLA-DQA2 gene, the HLA-DQB1 gene, the HLA-DQB2 gene, the HLA-DRA gene, the HLA-DRB1 gene, the HLA-DRB3 gene, the HLA-DRB4 gene, and the HLA-DRB5 is deleted.

19. A method of preparing a hypoimmunogenic stem cell, comprising:
reducing expression of at least one of an HLA-A gene, an HLA-B gene, an HLA-C gene, a β-Microglobulin (B2M) gene, a CIITA gene, a CD74 gene, a TAP1 gene, a MIC-1 gene, and a MIC-2 gene in a stem cell; and
conditionally expressing at least one of a CD24 gene, an HLA-G gene, and an HLA-E gene in the stem cell;
wherein the reducing the expression of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene and conditionally expressing the at least one of the CD24 gene, the HLA-G gene, and the HLA-E gene results in the hypoimmunogenic stem cell.

20. The method of claim 19, further comprising increasing expression of at least one of a CD46 gene, a CD59 gene, and a CD55 gene in the stem cell.

21. The method of claim 19, wherein reducing expression of at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene comprises deletion of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene a genomic DNA of the stem cell.

22. The method of claim 21, wherein the deletion of the at least one of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene from the genomic DNA of the stem cell comprises use of an endonuclease.

23. The method of claim 19, further comprising reducing expression of the HLA-A gene, the HLA-B gene, the HLA-C gene, the β-Microglobulin (B2M) gene, the CIITA gene, the CD74 gene, the TAP1 gene, the MIC-1 gene, and the MIC-2 gene in the stem cell.

24. The method of claim 19, wherein conditionally expressing at least one of the the CD24 gene, the HLA-G gene, and the HLA-E gene in the stem cell comprises inserting at least one of the CD24 gene, the HLA-G gene, and the HLA-E gene into a safe harbor locus of at least one allele of the stem cell.

25. The method of claim 24, wherein the safe harbor locus comprises an AAVS1 locus.

26. The method of claim 24, wherein conditionally expressing at least one of the CD24 gene, the HLA-G gene, and the HLA-E gene in the stem cell comprises over-expressing at least one of the CD24 gene, the HLA-G gene, and the HLA-E gene during a first cell-differentiation stage, and reducing expression of the at least one of the CD24 gene, the HLA-G gene, and the HLA-E gene during a second cell-differentiation stage relative to the first cell-differentiation stage.

27. The method of claim 24, wherein conditionally expressing at least one of the CD24 gene, the HLA-G gene, and the HLA-E gene in the stem cell comprises use of a pax7 promotor, a pax3 promotor, a myh4 promotor, a Ankrd1 promotor, a myod1 promotor, and myf5 promotor, or a myog promotor.

28. The method of claim 19, further comprising conditionally expressing the CD24 gene, the HLA-G gene, and the HLA-E gene in the stem cell.

29. The method of claim 19, further comprising reducing an expression of at least one of an HLA-DMA gene, an HLA-DMB gene, an HLA-DOA gene, an HLA-DOB gene, an HLA-DPA1 gene, an HLA-DPB1 gene, an HLA-DQA1 gene, an HLA-DQA2 gene, an HLA-DQB1 gene, an HLA-DQB2 gene, an HLA-DRA gene, an HLA-DRB1 gene, an HLA-DRB3 gene, an HLA-DRB4 gene, and an HLA-DRB5 gene in the stem cell.

30. A method of treating a subject in need thereof comprising administering to said subject a myogenic stem cell of claim 1.

31. The method of claim 30, wherein said subject suffers from a muscular disorder.

32. A composition comprising a myogenic stem cell of claim 1.
